(19) United States
(12) Patent Application Publication
(10) Pub. No.: US 2026/0183539 A1
(43) Pub. Date: Jul. 2, 2026

(54) ANCHOR FOR IMPLANTABLE STIMULATION DEVICE LEAD

(71) Applicant: Christopher Battista, Daniels Island, SC (US)

(72) Inventor: Christopher Battista, Daniels Island, SC (US)

(21) Appl. No.: 19/004,817

(22) Filed: Dec. 30, 2024

(51) Int. Cl. A61N 1/05; A61L 31/06

(52) U.S. Cl. CPC A61N 1/0558; A61L 31/06

(57) ABSTRACT

A lead anchor comprising a body formed into a generally tubular shape having holes at either end thereof and a slit extending longitudinally between the holes, so that the lead anchor may be opened and wrapped around a portion of an electrical lead wire. Anchor clips extend transversely around the lead anchor body and the lead wire, and the ends of the anchor clips penetrate surrounding subcutaneous tissue. When the lead anchors and electrodes are implanted into a patient's body, the anchor clips are crimped to secure the anchor body to the lead line, and to provide a gentle compressive force to the subcutaneous tissue disposed between the ends of the anchor clips.

### BACKGROUND OF THE INVENTION

In the medical field, tissue stimulation is a commonly used technique to treat and reduce pain in patients having various types of conditions. Electrical stimulation may be used as a physical therapy treatment that utilizes mild electrical pulses to help repair injured muscles, manipulate nerves, and reduce pain. In some cases, electrodes are implanted subcutaneously to target specific nerves, and steady streams of electrical pulses are delivered through the wires from the electrical stimulation unit in order to block the transmission of pain signals from reaching the spinal cord and brain. These pulses also stimulate the body to produce natural pain-relieving chemicals called endorphins.

Implantable stimulation devices often include an implantable control module with a pulse generator, a lead, and an array of stimulator electrodes that are implanted in contact with nerves, muscles, or other tissue to be stimulated. Electrical pulses are generated by the control module and delivered by the electrodes to body tissue. Typically, the electrode lead is anchored at various places in the body to prevent or reduce movement of the electrodes to prevent tissue damage, and to maintain contact between the electrodes and the targeted tissue, muscle or nerves.

Various efforts have been made to provide anchor mechanisms that are used to maintain the lead in place during the electrical stimulation treatment. The following documents are examples of various types of anchor mechanisms and systems that have been developed, and all of the documents cited below are hereby incorporated herein by reference, in their entireties:

U.S. Pat. No. 6,473,654 Lead Anchor

An anchor is provided for securing an elongated cylindrical member, such as a lead cable, to surrounding tissue. The anchor has a simple design that allows the anchor to be held in place on, e.g., a lead cable, without the need for sutures. In addition, the anchor may be manipulated to allow the anchor to move along the cylindrical member. Manipulation of the anchor is straight-forward, simple, and does not require tools, yet the anchor is reliably held in place, requiring intentional manipulation to be released. The anchor includes two coaxial sleeves holding the ends of a coaxial spring. As one sleeve is rotated in relation to the other, the spring is twisted, causing the inner diameter of the spring to increase or decrease. Once the inner diameter of the spring is increased to the point that is becomes larger than the outer diameter of the cylindrical member it surrounds, the anchor can slide on the cylindrical member. By decreasing the inner diameter of the spring, the spring can grip the cylindrical member, thus securing the anchor in place on the cylindrical member. The anchor includes a locking mechanism for holding the sleeves, and thus the spring, in a certain position or positions.

U.S. Pat. No. 7,899,553 Lead Anchor for Implantable Stimulation Devices

Disclosed is a lead anchor comprising a body made of an elastomeric material and defining a first opening and a second opening through which a lead can pass, one or more fasteners disposed within the body, with the ends of the fasteners protruding from the body, wherein the ends are configured and arranged to be clamped down to secure a lead passing through the body.

U.S. Pat. No. 9,079,019 Apparatus and Method for Anchoring Electrode Leads for Use with Implantable Neuromuscular Electrical Stimulator

Apparatus and methods for tethering an electrode lead to an anatomical structure within a patient using a coupling member are provided. An anchor configured to be secured to the anatomical structure and an electrode lead suitable for neuromuscular stimulation of spinal muscles and/or nerves innervating one or more muscles that contribute to spine stability may be used. The electrode lead is configured to be coupled to the anchor via the coupling member by securing a first end of the coupling member to the electrode lead and securing a second end of the coupling member to an eyelet of the anchor to place the electrode lead at a desired anatomical site within the patient.

U.S. Pat. No. 9,204,842 Medical Device Fixation Attachment Mechanism

A fixation member configured to anchor an implantable medical device within a patient is attached to an implantable medical device by introducing at least a portion of the fixation member in a tube mechanically connected to the medical device, and plastically deforming the tube in order to pinch the fixation member within a hollow space of the tube.

U.S. Pat. No. 9,492,657 Method of Implanting a Medical Device Including a Fixation Element

A system comprises an implantable medical device and an actively deployable clip attached to the implantable medical device that restricts movement of the implantable medical device once the clip is deployed within a body of a patient. In one embodiment, the implantable medical device is a lead and a clip that includes electrically conductive portion is an electrode of the lead. The implantable medical device may be implanted proximate to any suitable tissue site within the patient, and in one embodiment, the implantable medical device is implanted proximate to an occipital nerve or a trigeminal nerve of the patient.

U.S. Pat. No. 9,669,210 Electrical Stimulation Leads and Systems with Folding Anchoring Units and Methods of Making and Using

An electrical stimulation lead includes at least one anchoring unit and each anchoring unit includes a lead attachment element. Some anchoring units include one or more anchoring fins attached to the lead attachment element and extending away from the lead attachment element when in a deployed position for contact with patient tissue to anchor the lead within the patient tissue. Each anchoring fin also has a retracted position in which the anchoring fin folds down and lies next to the lead attachment element. Other anchoring units include one or more anchoring tabs defined by the lead attachment element. Each anchoring tab is partially separated from a remainder of the lead attachment element by at least one cutout and extends away from the remainder of the lead attachment element when in a deployed position for contact with patient tissue to anchor the lead within the patient tissue.

U.S. Pat. No. 10,112,045 Implantable Medical Device Fixation

An assembly includes an implantable medical device (IMD) including a conductive housing, and a fixation element assembly attached to the IMD. The fixation element assembly includes a set of active fixation tines and an insulator to electrically isolate the set of active fixation tines from the conductive housing of the implantable medical device. The active fixation tines in the set are deployable from a spring-loaded position in which distal ends of the active fixation tines point away from the implantable medical device to a hooked position in which the active fixation tines bend back towards the implantable medical device. The active fixation tines are configured to secure the implantable medical device to a patient tissue when deployed while the distal ends of the active fixation tines are positioned adjacent to the patient tissue.

U.S. Pat. No. 10,226,620 Anchor Elements, Medical Devices Including One or More Anchor Elements and Related Assemblies and Methods

Anchor elements include at least one protrusion configured to extend transversely from a longitudinal axis of the anchor element when the anchor element is in a deployed state. Anchor element assemblies and medical device assemblies may include such anchor elements. Methods of anchoring a medical device within a subject include securing at least a portion of the medical device within a lumen of at least one anchor element and deploying at least one protrusion of the at least one anchor element.

U.S. Pat. No. 10,561,835 Implantable Medical Lead with Threaded Fixation

The disclosure is directed to securing electrodes of a medical lead adjacent to a target tissue site. The medical lead may include one or more threaded fixation structures disposed circumferentially about the outer surface of the lead body, or elongated member, that resembles a “screw” or “auger.” During implantation, a clinician may rotate the entire lead to “screw” the lead into the tissue of the patient until electrodes of the lead reside adjacent to a target tissue. In this manner, the threaded fixation structure secures the lead within the patient to resist lead migration and improper therapy and provide a fine adjustment for depth of placement. The threaded fixation structure may be disposed on a portion of the lead proximal to or distal to the electrodes of the lead or over the portion of the lead that includes the electrodes.

U.S. Pat. No. 11,433,233 Electrode Contact for Subcutaneous Device

A subcutaneously implantable device includes a housing, a clip attached to the housing, a prong, and an electrode. The clip is configured to anchor the device to a muscle, a bone, and/or a first tissue. The prong has a base portion attached to the housing, an arm portion extending from the base portion so as to define a first plane that includes opposite ends of the arm portion of the prong and is perpendicular to a horizontal plane of the housing, and a contact portion that is configured to contact an organ, a nerve, the first tissue, and/or a second tissue. The contact portion is angled away from the first plane. The electrode is at the contact portion of the prong. The electrode is configured to contact the organ, the nerve, the first tissue, and/or a second tissue. Circuitry in the housing is in electrical communication with the electrode and is configured to provide monitoring, therapeutic, and/or diagnostic capabilities with respect to the organ, the nerve, the first tissue, and/or the second tissue.

### U.S. Application No. 20070100411 Implantable Medical Electrical Stimulation Lead Fixation Method and Apparatus

An implantable medical electrical lead for electrical stimulation of body tissue that includes a lead body extending between lead proximal and distal ends, at least one tine element that includes at least one flexible, pliant, tine, that is adapted to be folded inward and temporarily secured against the lead body using a temporary fixative, and at least one electrode, wherein the at least one electrode is distal of the at least one tine element on the lead body. A medical electrical stimulation system that includes an implantable pulse generator for providing medical electrical stimulation, and a medical electrical lead coupled to the implantable pulse generator for electrical stimulation of body tissue, the medical electrical lead including a lead body extending between lead proximal and distal ends. at least one tine element that includes at least one flexible, pliant, tine, that is adapted to be folded inward and temporarily secured against the lead body using a temporary fixative, and at least one electrode, wherein the at least one electrode is distal of the at least one tine element on the lead body.

### U.S. Application No. 20090082827 Hinged Anchors for Wireless Pacing Electrodes

A hinged anchor for a medical device electrode is disclosed. In one embodiment, the hinged anchor has a hinged portion and an anchor portion. The hinged portion can have a first configuration forming a first angle and a second configuration forming a second angle. The second angle can be a sharper angle than the first angle, and the hinged portion can be predisposed to assume the second configuration. The hinged anchor can be disposed on a control module of a leadless microstimulator device.

### U.S. Application No. 20100256696 Anchoring Units for Implantable Electrical Stimulation Systems and Methods of Making and Using

An anchoring unit for an implantable lead includes a body, a plurality of anchoring members, and at least one connecting element coupling together at least two of the anchoring members that are positioned adjacent to one another. The body is configured and arranged for positioning along a portion of an outer surface of a lead. The body has a first end, a second end, and a longitudinal axis extending therebetween. The first end is configured and arranged for placement on the lead so that the first end is positioned more distally on the lead than the second end. Each anchoring member has a proximal end and a distal end. The proximal end of each anchoring member extends from the body and the distal end of each anchoring member anchors to patient tissue upon implantation of the anchoring unit into the patient.

### U.S. Application No. 20100256696 Anchoring Units for Implantable Electrical Stimulation Systems and Methods of Making and Using

An anchoring unit for an implantable lead includes a body, a plurality of anchoring members, and at least one connecting element coupling together at least two of the anchoring members that are positioned adjacent to one another. The body is configured and arranged for positioning along a portion of an outer surface of a lead. The body has a first end, a second end, and a longitudinal axis extending therebetween. The first end is configured and arranged for placement on the lead so that the first end is positioned more distally on the lead than the second end. Each anchoring member has a proximal end and a distal end. The proximal end of each anchoring member extends from the body and the distal end of each anchoring member anchors to patient tissue upon implantation of the anchoring unit into the patient.

### U.S. Application No. 20130338730 Apparatus and Methods for Anchoring Electrode Leads Adjacent to Nervous Tissue

Apparatus for neuromuscular electrical stimulation and methods for anchoring the same are provided. The apparatus may include an elongated member having one or more electrodes disposed at the distal region of the elongated member and at least one fixation element disposed at the distal region of the elongated member. The fixation element may be shaped and sized to be deployed between tissue layers, such as muscle layers, without damaging the tissue layers so as to secure the one or more electrodes in or adjacent to a desired anatomical site within a patient. An additional fixation element may be disposed at the distal region of the elongated member so that tissue, such as a muscle, may be sandwiched between the fixation elements without damaging the tissue.

U.S. Application No. 20210146122 Subcutaneous Device for Use with Remote Device

A subcutaneously implantable device includes a clip that is configured to anchor the subcutaneously implantable device to a muscle, a bone, and/or a first tissue, and a first prong with a proximal end secured adjacent to the clip and a distal end extending away from the clip that is configured to contact an organ, a nerve, and/or a second tissue. A first electrode on the first prong is configured to contact the organ, the nerve, and/or the second tissue. A cable is conductively coupled to the first electrode, wherein the cable is configured to be connected to a remote device to electrically couple the subcutaneously implanted device to the remote device.

### U.S. Application No. 20230102674 Medical Device Lead Tip Anchor

Systems and methods which provide retractable anchor configurations for medical device leads are described. A retractable anchor may implement a retractable distention composed of a resilient material. The retractable distention may be distended when in a neutral state and may be contracted when in a biased state. A biasing bulkhead may be configured to receive a bias force sufficient to retract the retractable distention. A stylet may be inserted into an axial lumen of a medical device lead having retractable tip anchor structure and may engage the biasing bulkhead to apply a bias force. A stylet knob may be configured to interface with the stylet and provide bias force to be transferred to the biasing bulkhead of the retractable tip anchor structure. Locking the stylet knob on the medical device lead may maintain the bias force applied to the biasing bulkhead until the stylet knob is unlocked.

Wipo Publication No. WO2009135075 Techniques for Placing Medial Leads for Electrical Stimulation of Nerve Tissue

This disclosure is directed to extra, intra, and transvascular medical lead placement techniques for arranging medical leads and electrical stimulation and/or sensing electrodes proximate nerve tissue within a patient. It discloses an implantable medical lead system configured to deliver electrical stimulation to nerve tissue within a patient, the system comprising: an implantable medical lead comprising a distal portion configured for introduction into a sheath of tissue that contains the nerve tissue; an electrode electrically connected to the distal portion of the implantable medical lead; and an anchor connected to the medical lead and proximally offset from the electrode at least partially outside of the sheath to stabilize placement of the distal portion of the lead within the sheath.

### SUMMARY OF THE INVENTION

In one embodiment, the present invention includes a lead anchor comprising an anchor body (also referred to herein as a lead anchor), preferably made of a strong, biocompatible, lightweight material such as silicone that is in a generally tubular shape having an opening along one side, so that the lead anchor may be attached around a lead wire. In some embodiments, the anchor body may include a series of transversely oriented ribs that extend about the anchor body. This configuration somewhat resembles a hot dog bun, and allows the lead anchor to be opened so that a lead wire may be placed therein (similarly to placing a hot dog into a bun). Once the lead wire has been inserted into the lead anchor through the opening along the side thereof, the lead anchor resumes its generally tubular shape around the lead wire.

An anchor clip is used to secure the lead anchor and the electrode into place by encircling the anchor body and gently compressing the subcutaneous tissue adjacent to the position where the anchor is implanted into the body. Essentially, during the implant procedure, a doctor or medical professional inserts the electrode lead, along with the anchor body positioned around the lead wire, into a patient's body in the appropriate location and position, and then uses a crimping tool to crimp the anchor clip around the anchor, while simultaneously gently compressing the adjacent subcutaneous tissue to hold the anchor, lead and electrode in the proper desired position.

Yet another embodiment of the present invention is a method where a paddle-type implant is inserted over the spinal nerves after a laminectomy has been performed. This paddle implant has two independent leads extending distally that would subsequently be affixed to the soft tissues where one or more anchor clips are crimped around the anchor lead securing it to prevent migration of the paddle and leads.

### BRIEF DESCRIPTION OF THE DRAWINGS

These and other features, aspects, and advantages of the present invention will become better understood with regard to the following description, appended claims, and accompanying drawings where:

FIG. 1 is a perspective view that illustrates several examples of existing anchors for implantable stimulation electrode devices, whereby the anchor is sutured in place within surrounding subcutaneous tissue to temporarily secure the stimulation lead within a patient's body;

FIG. 2 is a perspective view of one embodiment of a crimping tool that is used to crimp an anchor clip of the present invention around an anchor body and lead wire, wherein the ends of the anchor clip extend beyond the anchor body so that the ends thereof may engage the surrounding subcutaneous tissue, causing the ends of the anchor clip to gently compress the subcutaneous tissue therebetween;

FIG. 3 is a partial top view of one embodiment of a crimping tool, showing the crimping mechanism and an anchor clip disposed within the crimping mechanism for placement about an anchor body;

FIG. 4 is an enlarged perspective view of one embodiment of an anchor body having a series of ribs that are disposed transversely about the anchor body, and further illustrating a longitudinal slit for receiving a lead wire;

FIG. 5 is a perspective view of one embodiment of an anchor body disposed about a lead wire;

FIG. 6 is a perspective view of one embodiment of an anchor body disposed about a lead wire, and further illustrating a crimping mechanism having an anchor clip disposed therein for crimping the anchor clip about an outer periphery of the anchor body, wherein the ends of the anchor clip extend beyond the anchor body so that the ends thereof may engage the surrounding subcutaneous tissue, causing the ends of the anchor clip to gently compress the subcutaneous tissue therebetween;

FIG. 7 is a perspective view of one embodiment of an anchor body disposed about a lead wire, and further illustrating a crimping mechanism having an anchor clip disposed therein, wherein the crimping operation around the anchor clip is in progress about an outer periphery of the anchor body, so that the ends of the anchor clip extend beyond the anchor body so that the ends thereof may engage the surrounding subcutaneous tissue, causing the ends of the anchor clip to gently compress the subcutaneous tissue therebetween;

FIG. 8 is a perspective view of one embodiment of an anchor body disposed about a lead wire, and further illustrating a crimping mechanism having an anchor clip disposed therein, wherein the crimping operation around the anchor clip is fully engaged about an outer periphery of the anchor body, so that the ends of the anchor clip extend beyond the anchor body so that the ends thereof may engage the surrounding subcutaneous tissue, causing the ends of the anchor clip to gently compress the subcutaneous tissue therebetween; and

FIG. 9 is a perspective view of one embodiment of an anchor body disposed about a lead wire, wherein an anchor clip extends about an outer periphery of the anchor body, thereby securing the anchor body to the lead wire, and further illustrating the distal ends of the anchor clip extending away from the anchor body to gently compress the subcutaneous tissue therebetween.

### DETAILED DESCRIPTION OF THE INVENTION

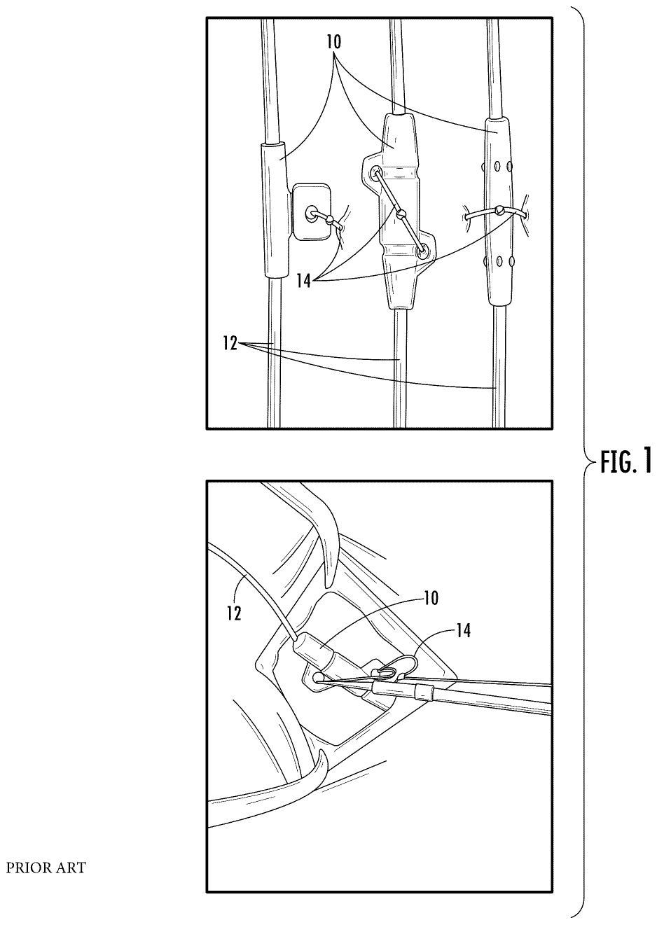

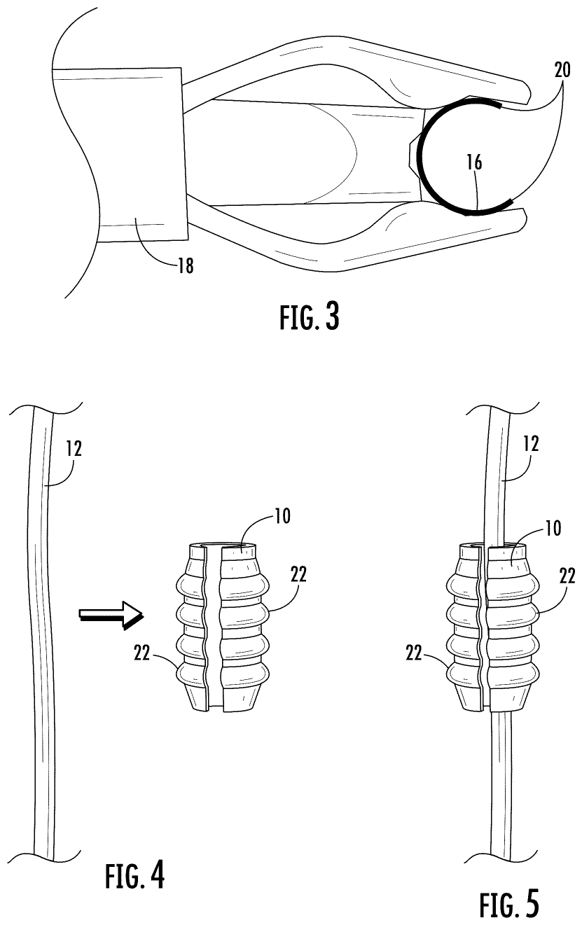

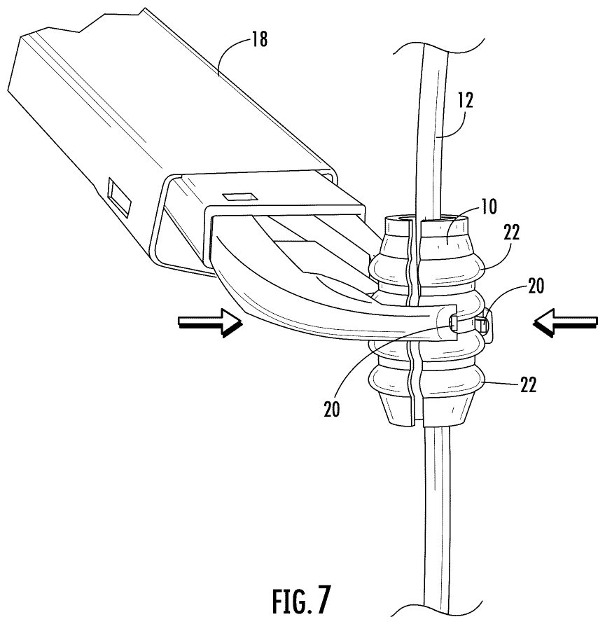

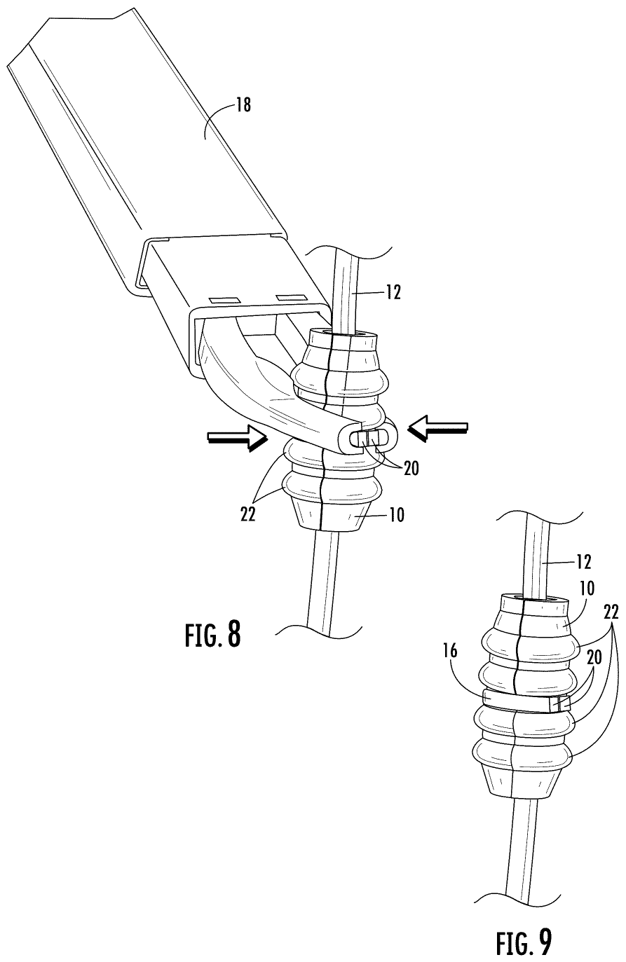

Heretofore, in order to permanently attach a subcutaneous electrical stimulation electrode to a person's tissue, an anchor sleeve or body 10 was disposed about the wire 12 adjacent the lead, and sutures 14 were used to stitch the anchor body 10 to the surrounding tissue, and several examples of these existing anchor mechanisms are shown in FIG. 1. However, the process for suturing the anchor sleeve to the surrounding tissue is tedious and time consuming, and if performed improperly, may culminate in the implanted lead migrating to an undesirable position within the spinal canal, which may ultimately require revision surgery. To address this issue, among others, the present invention, in a first embodiment, includes a lead anchor comprising an anchor body 10, preferably made of a strong, biocompatible, lightweight material such as silicone that is in a generally open-ended tubular shape having an opening along one side, so that the anchor body 10 may be attached around a lead wire 12, as shown in FIGS. 4-9. This configuration somewhat resembles a hot dog bun, and allows the anchor body 10 to be opened so that a lead wire 12 may be placed therein (similarly to placing a hot dog into a bun), as shown in FIGS. 3 and 4. Once the lead wire 12 has been inserted into the anchor body 10 through the opening along the side thereof, the anchor body 10 resumes its generally tubular shape around the lead wire 12, which extends through either end of the anchor body 10, as shown in FIGS. 4 and 5.

An anchor clip 16 is used to secure the anchor body 10 and the electrode into place by encircling the anchor body 10 and to provide gentle compression to the subcutaneous tissue adjacent to the position where the anchor body 10 is implanted into a patient's body. The anchor clip 16 is preferably applied to the anchor body 10 and lead wire 12 via a crimping tool 18, as shown in FIGS. 3 and 6-9, and the ends 20 of the anchor clip 16 are designed to extend beyond the anchor body 10, wherein the ends 20 of the anchor clip 16 may engage the surrounding subcutaneous tissue so that the ends 20 of the anchor clip 16 may gently compress the subcutaneous tissue therebetween. The anchor clips 16 are used to attach the anchor body 10 to tissue at the intended stimulation site and may also provide a controlled compressive load against the lead wire 12, thereby gripping the lead wire 12 to keep the lead wire 12 in place and prevent migration of the lead wire 12 away from the intended stimulation site.

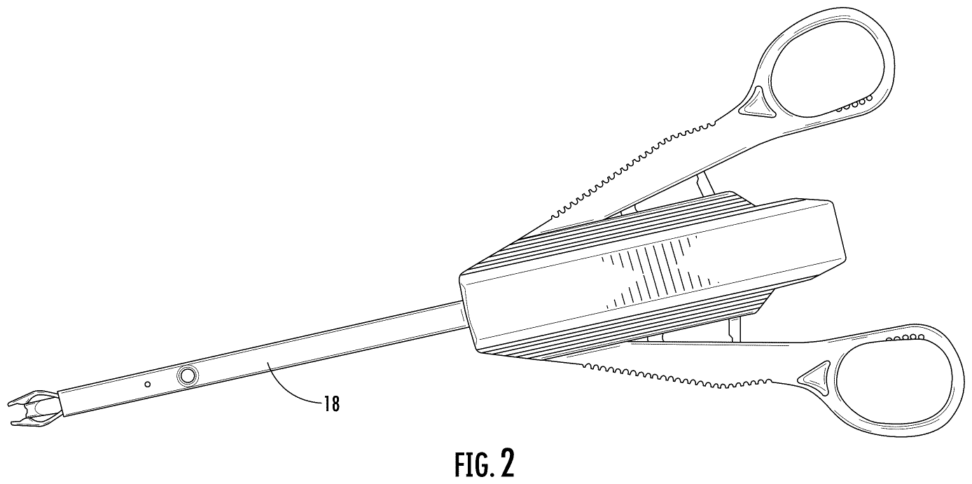

Specialized medical crimping tools 18 may be used to crimp the anchor clip around the anchor body and lead wire, as shown in FIGS. 2-3 and 6-9.

The anchor body 10 may be made of any elastomeric material suitable for implantation into a patient's body. The material is preferably compressible so as to compress against the lead when the anchor clips 16 are in the closed position. In some embodiments, the anchor body 10 is made of silicone, polyurethane, or a combination thereof. The anchor clips 16 may be made of any material that is suitable for implantation into a patient's body. In a preferred embodiment, the anchor clips 16 are made of any metal suitable for implantation into a patient's body.

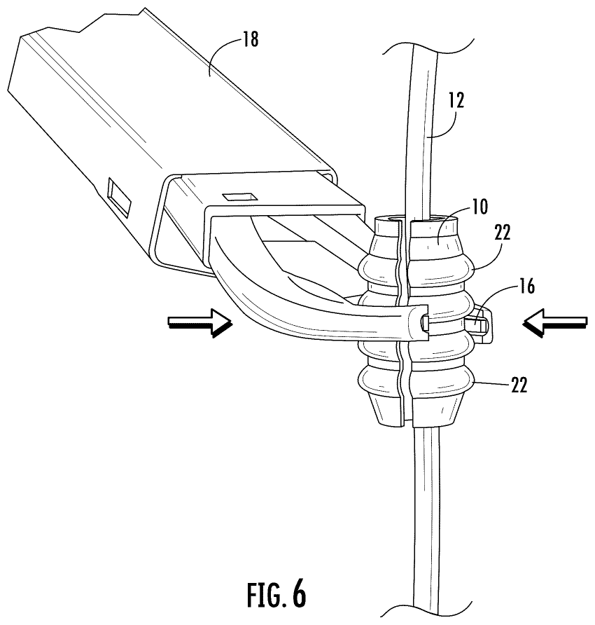

Essentially, during the implant procedure, a doctor or medical professional inserts the electrode lead wire 12, along with the anchor body 12 positioned around the lead wire 12, into a patient's body in the appropriate location and position, and then uses a crimping tool 18 to crimp the anchor clip 16 around the anchor body 10, while simultaneously gently compressing the adjacent subcutaneous tissue to hold the anchor body 10, lead wire 12 and electrode in the proper desired position, as shown in FIGS. 6-9. The implantable stimulation device typically includes a control module, an electrode array of stimulator electrodes, a lead coupling the control module to the electrode array, and one or more lead anchors. The control module also preferably includes a pulse generator that provides pulses of stimulation current to electrodes of the electrode array.

It should be noted that in most embodiments, the anchor clips 16 are only applied to the anchor body 10 and subcutaneous tissue at the time of implantation of the electrodes into a patient's body. Prior to implantation, the anchor clips 16 remain separate from the anchor body 10. In some embodiments, the ends 20 of the anchor clips 16 may be formed into sharp points for penetrating the subcutaneous tissue for gentle compression as a result of the crimping operation.

In an alternate embodiment, the crimping tool may include a type of ‘feeder magazine’ for holding a series of anchor bodies 10, so that the feeder magazine advances an anchor body 10 for placement about a lead wire, along with an anchor clip to be crimped about the anchor body. In this way, the crimping tool may include feeder magazines for both anchor bodies and anchor clips, so that each time a user employs the crimping tool to apply the anchor body and anchor clip to a lead wire, the feeder magazines advance the next anchor body and anchor clip to a ready position for the next crimping operation. These feeder magazines operate in similar principle to staples in a stapler, wherein after a staple is applied to paper, the next staple advances to the ready position for the next stapling operation. It is contemplated that separate feeder magazines may be used for the anchor bodies and anchor clips, or that a single feeder magazine may be configured to simultaneously and automatically advance an anchor body and an anchor clip for the next crimping operation.

Additionally, in some embodiments, the anchor body 10 may include a series of external ribs 22, which help to maintain the position of the anchor body 10 within the subcutaneous tissue, and serve to reduce movement of the anchor body 10 within the subcutaneous tissue while implanted therein due to the frictional engagement between the ribs and the surrounding subcutaneous tissue. The anchor clip 16 is preferably disposed between the ribs 22, in order to prevent the anchor body 10 from movement once the anchor clip 16 is crimped and engaged with the surrounding tissue, as shown in FIG. 9.

Although the present invention has been described in considerable detail with reference to certain preferred versions thereof, other versions are possible. Therefore, the spirit and scope of the appended claims should not be limited to the description of the preferred versions contained herein. All features disclosed in this specification may be replaced by alternative features serving the same, equivalent or similar purpose, unless expressly stated otherwise. Thus, unless expressly stated otherwise, each feature disclosed is one example only of a generic series of equivalent or similar features.

1. A lead anchor comprising:
a generally tubular shaped anchor body defining a first hole at a first end thereof and a second hole at a second end thereof, and a slit that extends longitudinally from said first hole to said second hole along said anchor body; and
an anchor clip having a first end and a second end, wherein said anchor clip extends around said anchor body in a transverse direction so that said first and second ends extend radially away from said anchor body for penetrating and providing compression of subcutaneous tissue that is disposed between said first and second ends of said anchor clip.

2. The lead anchor set forth in claim 1, wherein said first and second ends of said anchor clip are formed into sharpened points for penetrating said subcutaneous tissue.

3. The lead anchor set forth in claim 1, wherein said anchor body includes a series of external ribs extending about said anchor body in a transverse direction.

4. The lead anchor set forth in claim 1, further including an electrode lead wire that is positioned within said anchor body so that said anchor body surrounds a portion of said electrode lead wire.

5. The lead anchor set forth in claim 1, wherein said anchor body is made from material selected from the group consisting of silicone, polyurethane, and a combination thereof.
